SEQUENCE LISTING (12 sequences)

SEQ ID NO: 1 - DNA, 9528 bp, Homo sapiens
  CDS encoding Human NCAG1 protein (1507)..(5142)
  modified_base (1351): a, t, c, g, other or unknown
acctgctttcggccccgccccgcccgccgccggcctgctcacggctcctcccgtcctccccgaagccccgcctctgaccccgccctgtcctgtctccgtcccgccccacgcccgccagccagcgtcgctgtctctcgccttccctgaggccccgccttcagccccgccttcaaccccgccccgtcctgcctccgccccgcccccgcttgccggcccgcgtcgccgtctctcaccctccccgggctgcgcggccggagctggcacagaggatcctcggccgcggcgacatcaccgcctggggacgcgggcgctgctctggatacggcgccaccgagagaacccgccgcccgcgggtctctgtcctgcggtccgtggttgcccccacaagcgtccggcgtttcctgagggcgggcgtgtccgggccgtgcgggtcgcggggaccgagcgcggctgaggagaccgagcctggggcagcgcctgccgtagcgcgggagacgacgcgggggtcttgcggagccccgcgggagcctggcccgccgtgcagagcagttttctggaactctccacctccgtctcccttggggcccagtgcggcgccgagcccccgtcgggatctgcctgagaaagtgtcatgaaaaaagagcagaagagagacctcactgttgctgaaaggggaattttctttcgcccgttggcggttacttcatgatcggacgagaagtatctaggtgactgaagatattccatttttatgtttgtacacatgaagctgataaaagaagatgtgaacatgatttctctttgtcataataggctgatgagtaagtaagcctgaaaaatatttgaaatgaaggcaagaattttgaatttttaaaaaccaactaagactttgatcacttgttgaggatgtttctctctcataaatgaaagaaaaacgtattcacaagacaagaagtataaaaagttgagaggaatgacaactgagtccactcactcgaagaatgtcagtacttcatcatcttctttgggcaaacatacacaaatgcatcatacatgtgtggtgagcttatcaccagtgatggttttctgtgctagaaatgactcttaatttgaattttggagtgctttttctctttttttacaatgtgtgttccaactctttgtgttaaatagatttaagtaaaggaggtaaatgctaaattcatagtgttttttacctgtatcacttccctgtgtattatggaaaaattagagattttaacgttattcaaagttttactggaagcaaaactgtgccagggacagagatatacaatttaagtttctctttttggcaactgcacttgcttanaatgtactgaatgtcagctggatttcacagcatatcagatttacagtctttgtcttatcaaggcctttactgtatgttttatactaaccagatgggaaacacattgagcatcatatctgacatgtatgcctaagggaggagctcccccatggatcatggcgttaatgtttacaggacatttactattcttagcattattgatgtttgctttctctacttttgaggaatctgtgagcaattattccgaatgggcagttttcacagatgatatagatcagtttaaaacacagaaagtgcaagatttcagacccaaccaaaagctgaagaaaagtatgcttcatccaagtttatattttgatgctggagaaatccaagcaatgagacaaaagtctcgtgcaagccatttgcatctttttagagctatcagaagtgcagtgacagttatgctgtccaacccaacatactacctacctccaccaaagcatgctgattttgctgccaagtggaatgaaatttatggtaacaatctgcctcctttagcattgtactgtttgttatgcccagaagacaaagttgcctttgaatttgtcttggaatatatggacaggatggttggctacaaagactggctagtagagaatgcaccaggagatgaggttccaattggccattccttaacaggttttgccactgcctttgactttttatataacttattagataatcatcgaagacaaaaatacctggaaaaaatatgggttattactgaggaaatgtacgagtattccaaggtccgctcatggggcaaacagcttctccataaccaccaagccactaatatgatagcattactcacaggggccttggtgactggagtagataaaggatctaaagcaaatatatggaaacaggctgtagtggatgtcatggaaaagacaatgtttctattgaatcatattgttgatggttctttggatgaaggtgtggcctatggaagctacacagctaaatccgtcacacagtatgtttttctggcccagcgccattttaatatcaacaacttggataataactggttaaagatgcacttttggttctattatgccacccttttacctggcttccaaagaactgtgggtatagcagattccaattataattggttttatggtccagaaagccagctagttttcttggataagttcatcttaaagaatggagctggaaattggttagctcagcaaattagaaagcaccgacctaaagatggaccgatggttccttcaactgcccaaaggtggagtactcttcacactgaatacatctggtatgatccccagctcacaccacagccacctgctgattatggtactgcaaaaatacacacattccctaactggggtgtggttacttatggggctgggttgccaaacacacagaccaacacctttgtgtcttttaaatctgggaagctggggggacgagctgtgtatgacatagttcattttcagccatattcctggattgatgggtggagaagttttaacccaggacatgagcatccagatcagaactcatttacttttgcccccaatggacaagtatttgtttctgaagctctctatggacccaagttgagccaccttaacaatgtattggtgtttgctccatcaccctcaagccagtgtaataagccctgggaaggtcaactgggagaatgtgcgcagtggcttaagtggactggcgaggaggttggtgatgcagctggggaaataatcactgcctctcaacatggggaaatggtatttgtgagtggggaagccgtgtctgcttattcttcagcaatgagactgaaaagtgtatatcgtgctttgcttctcttaaattcccaaactctgctagttgttgatcatattgagaggcaagaagattccccaataaattctgtcagtgccttctttcataatttggatattgattttaaatatatcccatataagtttatgaataggtataatggtgccatgatggatgtgtgggatgcacattacaaaatgttttggtttgatcatcatggcaatagtcccatggccagtatacaggaagcagagcaagctgctgaatttaaaaaacgatggactcaatttgttaatgttacttttcagatggaacccacaatcacaagaattgcatatgtcttttatgggccatatatcaatgtctccagctgcagatttattgatagttccaatcctggacttcagatttctctcaatgtcaataatactgaacatgttgtttctattgtaactgattaccataacctgaagacaagattcaattatctgggattcggtggctttgccagtgtggctgatcaaggccaaataacccgatttggtttgggcactcaagcaatagtaaagcctgtaagacatgataggattattttcccctttggatttaaatttaatatagcagttggattaattttgtgcattagcttggtgattttaactttccaatggcgtttttacctttcttttagaaaactaatgcgatggatattaatacttgttattgccttgtggtttattgagcttttggatgtgtggagcacttgtagtcagcccatttgtgcaaaatggacaaggacagaggctgagggaagcaagaagtctttgtcttctgaagggcaccacatggatcttcctgatgttgtcattacctcacttcctggttcaggagctgaaattctcaaacaactttttttcaacagtagtgattttctctacatcagggttcctacagcctacattgatattcctgaaactgagttggaaatcgactcatttgtagatgcttgtgaatggaaggtgtcagatatccgcagtgggcattttcgtttactccgaggctggttgcagtctttagtccaggacacaaaattacatttgcaaaacatccatctgcatgaacccaataggggtaaactggcccaatattttgcaatgaataaggacaaaaaaagaaaatttaaaaggagagagtctttgccagaacaaagaagtcaaatgaaaggcgcctttgatagagatgctgaatatattagggctttgaggagacacctggtttactatccaagtgcacgtcctgtgctcagtttaagcagtggaagctggacgttaaagcttcatttttttcaggaagttttaggagcttcgatgagggcattgtacatagtaagagaccctcgggcatggatttattcaatgttgtacaatagtaaaccaagtctttattctttgaagaatgtaccagagcatttagcaaaattgtttaaaatagagggaggtaaaggcaaatgtaacttaaattcgggttatgctttcgagtatgaaccattgaggaaagaattatcaaaatccaaatcaaatgcagtgtccctcttgtctcacttgtggctagcaaatacagcagcagccttgagaataaatacagatttgctgcctactagctaccagctggtcaagtttgaagatattgtgcattttcctcagaaaactactgaaaggatttttgcctttcttggaattcctttgtctcctgctagtttaaaccaaatattgtttgccacctctacaaaccttttttaccttccctatgaaggggaaatatcaccaactaatactaatgtttggaaacagaacttgcctagagatgaaattaaactaattgaaaacatctgctggactctgatggatcgcctaggatatccaaagtttatggactaaatgctgcaggtcagcagaaatttgcactaataatacttaccaacccactttgtggatatgaatcagaagagtttgtttattctttagtgtgtgtgtgtgtgtgcacgcgtgtatgtgttcagtgttgtttgcacagagagattgttttaaaaaatggcaccatatttggcctagcaggatttatttttatgtcatcacctcccttgcctttgtttctgaaaattttgtctgctaaaaagtttctgctacagagtggtagatgaagttatatcatggggtcaggggagatgggaaaattttaagtttttgtctaactccccttcatctgtaactgtgctaatctatctagagacctcaaacactgctaaaggccttgcaattgctgctttacccacgcatctcttgctttcaagatggactacaaaagttccttatccttttgaaaaggtcttctgacacacttatcttgcacaaagaaaaagaaaatttcttttactgtgtttaatgttcagtgatatcactgaggaaatggtgaaagctcctatcagaactataggatttcttctgggaaatacagatggaaatacagaatgaatatgtttttttgaggtcggaaactgactttaaaagcctccttgaagttttttacttagaaatataaggaataagtctttgaacaatctgggtggcaagggctggtagattattttagacatgattgtctgtttaaaactctcctttcactttttatcctccctggagctacagctgttcgccatcacatcactcccatcctatcctttctgtcactgtcaagcaaaacaatcagtagttactaatcgctgaactctcaatattgtggggcattttccccccagttgattaattttgcgttaaagactgacacagacttagaatcaaatttatttttctggaattaacactctgtgactcaaagtagtgccactgcagtgtctttttaaactggaaacagaattggaaaactgcctgacttatcttgcatccctttgaatgagtttacagactgccagtgtctgcaaaagttgaaagcaaatgggagatgatgtcagaggcatctgtttcctttaccatctgcatcttattataaatgtagtcgtcataaagtgtggtttattttattttggtaggctctgaaatcaaaatgctacgccattataagccagtggagtaattacaatgtattggatgaaaacataaggcagtgtggagacttgatgaaaatctctgtacagattgcagtcttcttcctgatgtttcaaactgtggttcccccaagctctctaacacttggaagtctgtcattctgacctagataaaagtggttctttctcagtagttattattatgtcaaaatgtgcctccagagtgataaagctctgtatatgttagattccagctaaacctaacttggctgtcatttttcttccattatagtgtgagtggagactgccccccctccccaacatattccttcccatatctctcatgattgtccctctgtaatttcaaaatgaatgaaattcatgtgaatgtaggttgagagggcactgaagacctgaatctacactagtaatctcaagaaagattattcattctatctcagagttaccggcaagcatataaaatgctacttggataatatctacatgaatattgcatgctacatggttgataacactatttccattattgggcagaatctcagtgtttactttcaattcctaggatatgtgatcgtgaatcagatcacatataaaaagtctggattgtcagtagtattagatctgatcaaggtaggaattacaattgcatgcaggtagcaagcaagaaagcagaaactactgttccctttattttaacattgtacagacaatacagaaatgtacctgttggcggccgggtgcagtggctcacgcctgtaatcccagcacttcgggaggccgaggcgggtggatcacgaggtcaggagatcaagaccatcctggctaacacggtgaaaccccgtctctactaaaaaaaaagtacaaaaaattagccgggcgtggtggcgggcacctgtagtcccagctacacgggaggctgaggcaggagaatggcatgaacctgggaggcagagcttgcagtgagtggagatgcgccactgcactccagcctgggcgacagagcgagactccgcctcaaaaaaaaaaaaaaaaaagaaaaaaagaaatgtacctgttggcaggagaaggccagatggagtatgtggagtaatagggaaagaagagttacagaaaatgaaaaagaaaatgagttacactgagaatgaatatgggaacacgtcattgatagcaaaagaaaggtacaggcttacgaaaatgatctttacaatgtatcccagctttcacccccacatggcaatgcagagttgtatttacttgtttctgtactcacctactcccaccccaagggaagattttagacatgaaccctactatttagttattctaaaatagaaagtttgctggagaaagcgtctactcacagattgttctgtaaggaatgttatgtatgggtgagcgggtgacacatccattgggtatgtatgcatgtgatggtgcctgagacccctgccttagaaacagaattcctaaggggattgactctcccagcatgttcccaggtcctgcacccttagggtgatctaggaaaattttaaatagcttctactcttatttttgttctttgaaataattaaaagagggattatcactatctgatacttctgaaagaaacacttacaaaatttcttatctgtaaaatccgtctttttctacattaacttccccaaacataggcctaattgagataattgcttttattataataataggattgaaattttaaaattttgaaaggacttattaattttgctgacaaaagtgaagtaacaaatataatgataattggctttttaaattttcaaacaacatagatttactcaagatgaaataaaaaggccatattcagagttgaatttaatgaaaactcagaggaaataggaaaatctgctcaggagaaagaagctaaatctgcatagatttagtttgtagaatttaatttaaaatttaaattttaacaaagtgatgacacaacaatatgtacgtttaggtgtggacaccaaaatattagacatttgattgtccttttacatagagaataactaataaatgcctgacaagaatgggacaatccttccttgtatcaaaattcccaggtcttgctacattgccctctgcaaatgtattcaaagaagaacctcctccaccacttacttttggttggcataattgttcagcaacgatttctgtacatcaccaagtatctttggcattcttggtatacaaagtatatcacaattttaagtgagtaaatattaatgataatttttgaattgctttgtttggcttgattaactttgatcagaaatagaaacgttttcatttgttgatttaggaaaaagcataaatagaatgcagtataacaccacttccaaaggtaaggatacctaacattctttttttttttttttttttttttggggatggagtctcactttgttgcccaggctggagtgcagtggtctgatctcggctcactgcaacctccgcctaccgggttcaagtgattctcctacgtcagcctcctgaatagctgggattacaggtgcacgccaccatgcttggctcatttttgtatttttagtagtgacagcgtttcaccacattggtcaggctggntctcaatctcttgacctggtgatctgcccacctgggcctcccaaaatgctgggattacaggcatgagccaccacacctggcaagggtacctgacattctaagatatcaagacacttaatatgtgggctattagctgcttatttaaatgttgaccaaattgtctgatatatctgattaatcatgatttcacttcatttcggaagaaaaattatccatatcatttttaaagacgcaaatgactttggatttttgcatagagtacaatagacacttcaaacaatagattctaacattctctgaaacacttgagatgtttgagctaccatttatatgggttatttatatttagtctaagtaacacatacatgtttaattgattctgttttcatggatagattcaactaagtcttccaagcaattaattttttgttcgtcgtcgtttttycttcatacgttatctagttatgcagcactggaaacagactgaagatcataaaccagttttatcagacctatgtgtaataagactcctgttaatacaaaaataaaaagctaaaagcaa

SEQ ID NO: 2 - protein, 1212 aa, Homo sapiens
MALMFTGHLLFLALLMFAFSTFEESVSNYSEWAVFTDDIDQFKTQKVQDFRPNQKLKKSMLHPSLYFDAGEIQAMRQKSRASHLHLFRAIRSAVTVMLSNPTYYLPPPKHADFAAKWNEIYGNNLPPLALYCLLCPEDKVAFEFVLEYMDRMVGYKDWLVENAPGDEVPIGHSLTGFATAFDFLYNLLDNHRRQKYLEKIWVITEEMYEYSKVRSWGKQLLHNHQATNMIALLTGALVTGVDKGSKANIWKQAVVDVMEKTMFLLNHIVDGSLDEGVAYGSYTAKSVTQYVFLAQRHFNINNLDNNWLKMHFWFYYATLLPGFQRTVGIADSNYNWFYGPESQLVFLDKFILKNGAGNWLAQQIRKHRPKDGPMVPSTAQRWSTLHTEYIWYDPQLTPQPPADYGTAKIHTFPNWGVVTYGAGLPNTQTNTFVSFKSGKLGGRAVYDIVHFQPYSWIDGWRSFNPGHEHPDQNSFTFAPNGQVFVSEALYGPKLSHLNNVLVFAPSPSSQCNKPWEGQLGECAQWLKWTGEEVGDAAGEIITASQHGEMVFVSGEAVSAYSSAMRLKSVYRALLLLNSQTLLVVDHIERQEDSPINSVSAFFHNLDIDFKYIPYKFMNRYNGAMMDVWDAHYKMFWFDHHGNSPMASIQEAEQAAEFKKRWTQFVNVTFQMEPTITRIAYVFYGPYINVSSCRFIDSSNPGLQISLNVNNTEHVVSIVTDYHNLKTRFNYLGFGGFASVADQGQITRFGLGTQAIVKPVRHDRIIFPFGFKFNIAVGLILCISLVILTFQWRFYLSFRKLMRWILILVIALWFIELLDVWSTCSQPICAKWTRTEAEGSKKSLSSEGHHMDLPDVVITSLPGSGAEILKQLFFNSSDFLYIRVPTAYIDIPETELEIDSFVDACEWKVSDIRSGHFRLLRGWLQSLVQDTKLHLQNIHLHEPNRGKLAQYFAMNKDKKRKFKRRESLPEQRSQMKGAFDRDAEYIRALRRHLVYYPSARPVLSLSSGSWTLKLHFFQEVLGASMRALYIVRDPRAWIYSMLYNSKPSLYSLKNVPEHLAKLFKIEGGKGKCNLNSGYAFEYEPLRKELSKSKSNAVSLLSHLWLANTAAALRINTDLLPTSYQLVKFEDIVHFPQKTTERIFAFLGIPLSPASLNQILFATSTNLFYLPYEGEISPTNTNVWKQNLPRDEIKLIENICWTLMDRLGYPKFMD

SEQ ID NO: 3 - DNA, 5092 bp, Mus sp.
  CDS encoding the mouse NCAG1 protein (501)..(4121)
tctgagaatgacagtactttatcatcttcttttggggaacatacagaaacataccatttatgtgtggtaagttaatcactacagatggtttcttgtgctacgtggtcaaatggcttcatttgaattttggaattttaaaaaattttttctttttcacatgttaattagatttacacacagggagtaaatgttggatttgttgtattttctgactagaccactgttttctgtgcattggagacattggaggcattaatattccttgaaattttattttattggaagcaaacctgtgccagggacacagacatgctatataatttcctaacttttcttgctttgaataagctgaatgtcacctggatttcacagcctatgaggtatagtctgttttttgtttttgtttttttgctacatctttaatatataatttacaataaccagatgggaaacactgtgcttaacacatatgcctaaggaaaagatcttccccatggatcatggcgtttatgtttacagaacatttactatttttaacattgatgatgtgtagtttttctacttgtgaagaatctgtgagcaattattctgaatgggcagttttcacagacgatatacaatggcttaagtcacagaaaatacaagatttcaaactcaaccgaagacttcatccaaatttatattttgatgctggagatatacaaacattgaaacaaaagtctcgtacaagccatttgcatatttttagagctatcaaaagtgcagtgacaattatgctgtccaatccatcatactacctacctccacccaagcatgctgagtttgctgccaagtggaatgaaatttatggtaataatcttcctcctttagcattgtattgtttattatgcccagaagacaaggttgcctttgaatttgttatggaatacatggatcggatggttagctacaaagactggctagttgagaatgcaccaggggatgaggttccagttggccattctttaacaggttttgccactgcctttgactttttatataatctattaggtaatcagcgtaaacaaaaatacctagaaaaaatttggattgttactgaggaaatgtatgaatattccaagattcgatcatggggcaaacaacttcttcataaccatcaagctacaaatatgatagctttactcataggggccttggttactggagtagataaaggatctaaagcaaacatatggaaacaagttgttgttgatgtgatggaaaagactatgtttctcttgaagcatattgtagatggctcattggatgaaggtgtggcctatggaagctatacctcaaaatcagttacacagtatgtttttttggcacaacgccattttaacatcaacaactttgataataactggctaaaaatgcatttttggttttattatgctacacttttgccaggctatcaaagaactgtaggcatagcagattccaattataattggttttatggtccagagagccagctagttttcttggataagttcattttacagaatggagctggaaattggttagctcagcaaattagaaagcatcgacctaaggatggaccaatggttccttccactgctcagcggtggagtactcttcatactgaatacatctggtatgatccaacactcaccccacagcctcctgttgattttggcactgcaaaaatgcacacatttcctaactggggtgtcgtgacttatgggggtgggctgccaaacacccagaccaatacctttgtgtcttttaaatctgggaaactgggaggacgagctgtgtatgacatagttcactttcagccatattcctggattgatggatggagaagctttaacccaggacatgaacatccagatcaaaattcatttactttcgctcctaatgggcaggtattcgtttctgaggctctttatggaccaaaattgagccaccttaacaacgtattggtgtttgccccatcaccatcaagtcaatgtaatcagccctgggaaggtcaactgggagaatgtgcacagtggctcaagtggactggggaagaggttggtgatgcagctggggaagttattactgctgctcaacatggtgataggatgtttgtgagtggggaagcagtgtctgcttattcttctgccatgagactgaaaagtgtctatcgtgctttacttcttttaaattcacaaactctgcttgttgtcgatcatattgaaaggcaagaaacttccccaataaattctgtcagtgccttctttcataatttggatattgattttaaatacatcccatacaagtttatgaatagatataatggtgccatgatggatgtgtgggatgcacactataaaatgttttggtttgatcaccatggcaacagtcctgtggctaatatacaggaagcagaacaggctgctgaatttaagaaacggtggacacagtttgttaatgttacatttcatatggaatccacaatcacaagaattgcttatgtattttatgggccatatgtcaatgtttccagctgcagatttattgatagttccagttctggacttcagatttctttacatgtcaacagtactgaacatagtgtgtctgttgtaactgactatcaaaaccttaaaagcagattcagttacctgggatttggtggttttgccagtgtggctaatcaaggacagataaccagatttggtttgggtactcaagaaatagtaaaccctgtaagacatgataaagttaatttcccctttgggtttaaatttaatatagcagttggattcattttgtgtattagtttggttattttaacttttcaatggcggttttacctttcctttagaaagctaatgcgctgtgtattaatacttgttattgccttgtggtttattgagcttctggatgtatggagtacatgcactcagcccatctgtgcaaaatggacaaggactgaagctaaggcaaatgagaaggtcatgatttctgaagggcatcatgtggatcttcctaatgttattattacctcactccctggttcaggagctgaaattctcaaacagctttttttcaacagcagtgattttctctacatcagaattcctacagcctacatggatatccctgaaactgaatttgaaattgactcatttgtagatgcttgtgagtggaaagtatcagatatccgcagtgggcactttcatcttcttcgagggtggctgcagtctttggtccaggatacaaaacttcacttgcaaaacatccatctacatgaaaccagtaggagtaaactggcccaatattttacaactaataaggacaaaaagcgaaaattaaaaagaagggagtctttgcaagatcaaagaagtagaataaaaggaccatttgatagagatgctgaatatattagggctttaagaagacaccttgtttattacccaagtgcacgtcctgtgctcagcttaagtagtggtagctggacattgaagcttcatttttttcaggaagttttaggaacttcaatgcgggcattgtacatagtaagagaccctcgagcttggatctattcagtgctatatggtagtaaaccaagtctttattctttgaagaatgtaccagagcacttagcaaaattgtttaaaatagaggaaggtaaaagcaaatgtaattcgaattctggctatgcttttgagtatgaatcactgaagaaagaattagaaatatcccaatcaaatgctatctccttattatctcatttgtgggtagcaaacactgcagcagccttgagaataaatacagatttgctgcctaccaattaccatctggtcaagtttgaagatattgttcattttcctcagaagactactgaaaggatttttgctttccttggcattcctttgtctcctgctagtttaaaccaaatgctatttgccacttccacaaaccttttttatcttccatatgagggggaaatatcaccatctaatactaatatttggaaaacaaacttgcctagagatgaaattaaactaattgaaaacatttgctggacactgatggatcatctaggatatccaaagtttatggactaaatgctgcaggtcggcaaaatttgcactaatgtgtcccaacctactttgtggatatgaactagaaaactttgtttattcttgtacatgtatgtatgtgtgtagagtgagtgcgtgtgtccagtatgttatttgcacagagatattttcaaaataggcaccatatttggcctagcaggatttatttttatgttaccacttttcttgcctttgtttctgaatttttttctgctaaaatgtttctgctacagaggtatatattctggggttctgaaatatggggttttaatggactttaactcaacttctttggaaactatttatctatcttaggacctcaaacactacaaacggccttgcaattgctgctgtatctagtcatctctcgctcttaatatggactacaaaactttatgttttgaaaacgtctaacatttaccttgcacacaaaaacgagaaataaaaaaacaaaaattattttacgttgtatagtgtttattgaaatcacttggtgaggctggggggaggagcttatgataaagttcccttaagaaactagaaaataaagatgaaaacatagaattaaggtttttttgtttctttcttccttttttttttttttttgtactaagaaataagattgaacagtggatactgaaatttggtgaattattttggaagtgattctctcatttgtctttctgaagctacagctgttcatcatcacactacccttaccctgtctatccattctgtcattgtcaccaaaaaaaaaaagtcagtaattactagctacaaaactatctaacaagcccttctctggatgatttactttgtgttaaagacttacacagatttataatcacatttagttgtgtggcattaccacaatatgactcaaagcaaaagcagacttctgtctgttgtagtgtttttaagtgtgtgttgtggggtgggggagggsrsdback

SEQ ID NO: 4 - protein, 1207 aa, Mus sp.
MAFMFTEHLLFLTLMMCSFSTCEESVSNYSEWAVFTDDIQWLKSQKIQDFKLNRRLHPNLYFDAGDIQTLKQKSRTSHLHIFRAIKSAVTIMLSNPSYYLPPPKHAEFAAKWNEIYGNNLPPLALYCLLCPEDKVAFEFVMEYMDRMVSYKDWLVENAPGDEVPVGHSLTGFATAFDFLYNLLGNQRKQKYLEKIWIVTEEMYEYSKIRSWGKQLLHNHQATNMIALLIGALVTGVDKGSKANIWKQVVVDVMEKTMFLLKHIVDGSLDEGVAYGSYTSKSVTQYVFLAQRHFNINNFDNNWLKMHFWFYYATLLPGYQRTVGIADSNYNWFYGPESQLVFLDKFILQNGAGNWLAQQIRKHRPKDGPMVPSTAQRWSTLHTEYIWYDPTLTPQPPVDFGTAKMHTFPNWGVVTYGGGLPNTQTNTFVSFKSGKLGGRAVYDIVHFQPYSWIDGWRSFNPGHEHPDQNSFTFAPNGQVFVSEALYGPKLSHLNNVLVFAPSPSSQCNQPWEGQLGECAQWLKWTGEEVGDAAGEVITAAQHGDRMFVSGEAVSAYSSAMRLKSVYRALLLLNSQTLLVVDHIERQETSPINSVSAFFHNLDIDFKYIPYKFMNRYNGAMMDVWDAHYKMFWFDHHGNSPVANIQEAEQAAEFKKRWTQFVNVTFHMESTITRIAYVFYGPYVNVSSCRFIDSSSSGLQISLHVNSTEHSVSVVTDYQNLKSRFSYLGFGGFASVANQGQITRFGLGTQEIVNPVRHDKVNFPFGFKFNIAVGFILCISLVILTFQWRFYLSFRKLMRCVLILVIALWFIELLDVWSTCTQPICAKWTRTEAKANEKVMISEGHHVDLPNVIITSLPGSGAEILKQLFFNSSDFLYIRIPTAYMDIPETEFEIDSFVDACEWKVSDIRSGHFHLLRGWLQSLVQDTKLHLQNIHLHETSRSKLAQYFTTNKDKKRKLKRRESLQDQRSRIKGPFDRDAEYIRALRRHLVYYPSARPVLSLSSGSWTLKLHFFQEVLGTSMRALYIVRDPRAWIYSVLYGSKPSLYSLKNVPEHLAKLFKIEEGKSKCNSNSGYAFEYESLKKELEISQSNAISLLSHLWVANTAAALRINTDLLPTNYHLVKFEDIVHFPQKTTERIFAFLGIPLSPASLNQMLFATSTNLFYLPYEGEISPSNTNIWKTNLPRDEIKLIENICWTLMDHLGYPKFMD

SEQ ID NO: 5 - DNA, 30 bp, Unknown Organism
  Description of Unknown Organism Illustrative oligonucleotide
ccgccgccgccgccgccgccgccgccgccg

SEQ ID NO: 6 - DNA, 30 bp, Unknown Organism
  Description of Unknown Organism Illustrative oligonucleotide
cggcggcggcggcggcggcggcggcggcgg

SEQ ID NO: 7 - DNA, 21 bp, Unknown Organism
  Description of Unknown Organism Illustrative oligonucleotide
ccgccgccgccgccgccgccg

SEQ ID NO: 8 - DNA, 18 bp, Unknown Organism
  Description of Unknown Organism Illustrative oligonucleotide
cggcggcggcggcggcgg

SEQ ID NO: 9 - DNA, 18 bp, Unknown Organism
  Description of Unknown Organism Illustrative oligonucleotide
ccgccgccgccgccgccg

SEQ ID NO: 10 - DNA, 12 bp, Unknown Organism
  Description of Unknown Organism Illustrative oligonucleotide
ccgccgccgccg

SEQ ID NO: 11 - DNA, 18 bp, Unknown Organism
  Description of Unknown Organism Illustrative oligonucleotide
cagcagcagcagcagcag

SEQ ID NO: 12 - DNA, 36 bp, Unknown Organism
  Description of Unknown Organism Illustrative oligonucleotide
cggcggcggcggcggcggcagcagcagcagcagcag

(19) United States
(12) Patent Application Publication
(10) Pub. No.: US 2005/0118581 A1
(43) Pub. Date: Jun. 2, 2005

(54) Novel brain expressed gene and protein associated with bipolar disorder

(76) Inventors: Jurgen, Peter, Lode Del-Favero, Tienen (BE); Christine Van Broeckhoven, Edegem (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/479,472

(22) PCT Filed: Jun. 6, 2002
(86) PCT No.: PCT/EP02/06316

(30) Foreign Application Priority Data
Jun. 11, 2001 (EP) 01202214.1

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

We previously identified 18q21.33-q23 as a candidate region for bipolar (BP) disorder and constructed a yeast artificial chromosome (YAC) contig map. In a next step we isolated and analysed all CAG/CTG repeats from this region and excluded them from involvement in BP disorder. Here, in the process of identifying all CCG/CGG repeats from the region, we isolated three potential CpG islands, one of which is located 1.5 kb upstream of a predicted exon of 3639 bp. Further analysis showed this was part of a novel CpG-associated, brain-expressed gene, that we called NCAGI (Novel CpG Associated Gene 1). Mutation analysis of this positional and functional candidate identified two single nucleotide polymorphisms, none of which were shown to be associated with the BP phenotype.

### FIELD OF THE INVENTION

The invention is broadly concerned with the determination of genetic factors associated with psychiatric health. More particularly, the present invention is directed to a human gene which is linked to a mood disorder or related disorder in affected individuals and their families. Specifically, the present invention is directed to a gene located on the eighteenth chromosome that is expressed in brain tissue and may be used as a diagnostic marker for bipolar disorder.

### BACKGROUND OF THE INVENTION

Pharmacogenetics Background:

Every individual is a product of the interaction of their genes and the environment. Pharmacogenetics is the study of how genetic differences influence the variability in patients responses to drugs. Through the use of pharmacogenetics, we will soon be able to profile variations between individuals'DNA to predict responses to a particular medicine. Target validation that will predict a well-tolerated and effective medicine for a clinical indication in humans is a widely perceived problem; but the real challenge is target selection. A limited number of molecular target families have been identified, including receptors and enzymes, for which high throughput screening is currently possible. A good target is one against which many compounds can be screened rapidly to identify active molecules (hits). These hits can be developed into optimized molecules (leads), which have the properties of well-tolerated and effective medicines. Selection of targets that can be validated for a disease or clinical symptom is a major problem faced by the pharmaceutical industry. The best-validated targets are those that have already produced well-tolerated and effective medicines in humans (precedent targets). Many targets are chosen on the basis of scientific hypotheses and do not lead to effective medicines because the initial hypotheses are often subsequently disproved.

Two broad strategies are being used to identify genes and express their protein products for use as high-throughput targets. These approaches of genomics and genetics share technologies but represent distinct scientific tactics and investments. Discovery genomics uses the increasing number of databases of DNA sequence information to identify genes and families of genes for tractable or scrollable targets that are not known to be genetically related to disease.

The advantage of information on disease-susceptibility genes derived from patients is that, by definition, these genes are relevant to the patients'genetic contributions to the disease. However, most susceptibility genes will not be tractable targets or amenable to high-throughput screening methods to identify active compounds.

The differential metabolism related to the relevant gene variants can be studied in focused functional genomic and proteomic technologies to discover mechanisms of disease development or progression.

Critical enzymes of receptors associated with the altered metabolism can be used as targets. Gene-to-function-to-target strategies that focus on the role of the specific susceptibility gene variants on appropriate cellular metabolism become important.

Data mining of sequences from the Human Genome Project and similar programmes with powerful bioinformatic tools has made it possible to identify gene families by locating domains that possess similar sequences. Genes identified by these genomic strategies generally require some sort of functional validation or relationship to a disease process. Technologies such as differential gene expression, transgenic animal models, proteomics, in situ hybridization and immunohistochemistry are used to imply relationships between a gene and a disease.

The major distinction between the genomic and genetic approaches is target selection, which genetically defined genes and variant-specific targets already known to be involved in the disease process. The current vogue of discovery genomics for nonspecific, wholesale gene identification, with each gene in search of a relationship to a disease, creates great opportunities for development of medicines.

It is also critical to realize that the core problem for drug development is poor target selection. The screening use of unproven technologies to imply disease-related validation, and the huge investment necessary to progress each selected gene to proof of a concept in humans, is based on an unproven and cavalier use of the word ‘validation’. Each failure is very expensive in lost time and money. For example, differential gene expression (DGE) and proeomics are screening technologies that are widely used for target validation. They detect different levels and/or patterns of gene and protein expression in tissues, which may be used to imply a relationship to a disease affecting that tissue.

Mood Disorder Background:

Mood disorders or related disorders include but are not limited to the following disorders as defined in the Diagnostic and statistical Manual of Mental Disorders, version 4 (DSM-W) taxonomy DSM-IV codes in parenthesis): mood disorders (296.XX,300.4,311,301.13,295.70), schizophrenia and related disorders (295.XX,297.1,298.8,297.3,298.9), anxiety disorders (300.XX,309.81,308.3), adjustment disorders (309.XX) and personality disorders (codes 301.XX).

The present invention is particularly directed to genetic factors associated with a family of mood disorders known as Bipolar (BP) spectrum disorders. Bipolar disorder (BP) is a severe psychiatric condition that is characterized by disturbances in mood, ranging from an extreme state of elation (mania) to a severe state of dysphoria (depression). Two types of bipolar illness have been described: type I BP illness (BPI) is characterized by major depressive episodes alternated with phases of mania, and type II BP illness (BPII), characterized by major depressive episodes alternating with phases of hypomania. Relatives of BP probands have an increased risk for BP, unipolar disorder (patients only experiencing depressive episodes; UP), cyclothymia (minor depression and hypomania episodes; cy) as well as for schizoaffective disorders of the manic (SAm) and depressive (SAd) type. Based on these observations BP, cY, UP and SA are classified as BP spectrum disorders.

The involvement of genetic factors in the etiology of BP spectrum disorders was suggested by family, twin and adoption studies (Tsuang and Faraone (1990), the Genetics of Mood Disorders, Baltimore, The John Hopkins University Press) However, the exact pattern of transmission is unknown. In some studies, complex segregation analysis supports the existence of a single major locus for BP (Spence et al. (1995), Am J. Med. Genet (Neuropsych. Genet.) QQ pp 370-376). Other researchers propose a liability-threshold-model, in which the liability to develop the disorder results from the additive combination of multiple genetic and environmental effects (McGuffin et al. (1994), Affective Disorders; Seminars in Psychiatric Genetics Gaskell, London pp 110-127).

Due to the complex mode of inheritance, parametric and non-parametric linkage strategies are applied in families in which BP disorder appears to be transmitted in a Mendelian fashion. Early linkage findings on chromosomes 11p15 (Egeland et al. (1987), Nature˜pp 783-787) and Xq27-q28 (Mendlewicz et al. (1987, the Lancet 1 pp 1230 -1232; Baron et al. (1987) Nature 12 & pp 289-292) have been controversial and could initially not be replicated (Kelsoe et al. (1989) Nature˜pp 238-243; Baron et al. (1993) Nature Genet˜pp 49-55) with the development of a human genetic map saturated with highly polymorphic markers and the continuous development of data analysis techniques, numerous new linkage searches were started. In several studies, evidence or suggestive evidence for linkage to particular regions on chromosomes 4, 12, 18, 21 and X was found (Black wood et al. (1996) Nature Genetics˜pp 427-430, Craddock et al. (1994) Brit J. psychiatry˜pp355-358, Berrettini et al. (1994), Proc Natl Acad Sci USA˜pp 5918-5921, Straub et al. (1994) Nature Genetics˜pp 291-296 and Pekkarinen et al. (1995) Genome Research 2 pp 105-115). In order to test the validity of the reported linkage results, these findings have to be replicated in other, independent studies.

Recently, linkage of bipolar disorder to the pericentromeric region on chromosome 18 was reported (Berrettini et al. 1994). Also a ring chromosome 18 with break-points and deleted regions at 18pter-p11 and 18q23-qter was reported in three unrelated patients with BP illness or relates syndromes (Craddock et al. 1994). The chromosome 18p linkage was replicated by stine et al. (1995) Am J. Hum Genet 22 pp 1384-1394, who also reported suggestive evidence for a locus on 18q21.2-q21.32 in the same study.

Interestingly, Stine et al. observed a parent-of-origin effect: the evidence of linkage was the strongest in the paternal pedigrees, in which the proband's father or one of the proband's father's sibs is affected. Several studies described anticipation in families transmitting BP disorder(McInnis et al 1993, Nylander et al 1994) suggesting the involvement of trinucleotide repeat expansions (TREs), considering a number of diseases caused by an expansion of a CAG/CTG, a CCG/CGG or a GAAJTTC repeat show anticipation (reviewed by Margolis et al.(Margolis et al 1999)). Previous efforts to find potentially expanded repeats have primarily focused on CAG/CTG repeats although the search for CCG/CGG repeats is increasing(Kleiderlein et al 1998, Mangel et al 1998, Eichhammer et al 1998, Kaushik et al 2000). Previously, we reported on a new method for the region specific isolation of triplet repeats: triplet repeat YAC fragmentation(Del Favero et al 1999). This proved to be a valid method for the isolation of CAG/CTG repeats and using this method, we exlcuded the involvement of CAG/CTG repeats from within 18q21.33-q23 in bipolar disorder(Goossens et al 2000). The present invention adapted the method for the region specific isolation of CCG/CGG repeats and applied it to the chromosome 18q21.33-q23 BP candidate region.

### SUMMARY OF THE INVENTION

The present invention is directed to a novel gene and protein encoded by that gene.

The novel gene is located at an 8.9 cM chromosome region located between D18S68 and D18S979 at 18q21.33-q23 A physical map was constructed using yeast artificial chromosomes (YACs)(Verheyen et al 1999).

The previously described method was adapted for the region specific isolation of CCG/CGG repeats and applied to the chromosome 18q21.33-q23 BP candidate region. Three potential CpG islands were isolated, one of which is located 1.5 kb upstream of a predicted exon of 3639 bp. Further analysis showed this was part of a novel CpG-associated, brain-expressed gene, herein called NCAGI (Novel CpG Associated Gene 1). Mutation analysis of this positional and functional candidate identified two single nucleotide polymorphisms, which may be useful as a diagnostic marker for BP phenotype.

### BRIEF DESCRIPTION OF THE DRAWING

FIG. 1. List of all human ESTs found by BLASTN alignment searches of dbEST. ESTs are named with their Genbank Acc Nos. I.M.A.G.E. Consortium [LLNL] cDNA Clones(Lennon et al 1996) are named with their RZPD clone ID.

FIG. 2: Minimal YAC tiling path of the 18q21.33-q23 BP candidate region(Verheyen et al 1999). The YACs are represented by solid lines, the CCG/CGG fragmentation products by dotted lines. YAC sizes, between brackets, are estimated by PFGE analysis. Solid circles indicate positive STS/STR hits. Shaded boxes highlight the CCG/CGG repeat and the three CpG islands isolated by YAC fragmentation.

FIG. 3: Feature map of NCAG1. a) Predicted Features by bioinformatics. They encompass the CpG island as predicted by LCP(Huang 1994) and CPG(Larsen et al 1992), the ORF or exon as predicted by Grail(Uberbacher & Mural 1991) and Genscan(Burge & Karlin 1997), the transcription start site (TSS) as predicted by Proscan(Prestridge 1995)and the relevant polyadenylation signals as predicted by PolyAH(Salamov & Solovyev 1997). The numbers below the features indicate the scores as returned by Proscan and PolyAH. b) Alignment of EST hits. ESTs are named with their Genbank Acc Nos. c) Alignment of cDNA clones. I.M.A.G.E. Consortium [LLNL] cDNA Clones(Lennon et al 1996) are named with their RZPD clone ID. d) RT-PCR products. The grey bars represent the RT-PCR product, the thin black lines represent the sequences obtained on the nested PCRs.

### DETAILED DESCRIPTION OF THE INVENTION

The present invention is directed to a novel gene located at the 18q chromosomal candidate region of chromosome 18. More specifically, the gene is located at an 8.9 cM region located between DI 8S68 and DI 8S979 at 18q21.33-q23.

The gene is located at a chromosomal region associated with mood disorders such as bipolar spectrum disorders and may therefore be useful as a diagnostic marker for bipolar spectrum disorders. The region in question when removed from the totality of the human genome may also be used to locate, isolate and sequence other genes which influences psychiatric health and mood.

Isolation and Identification of Identification of Novel Gene:

Standard procedures well-known to one skilled in the art were applied to the identified YAC clones and, where applicable, to the DNA from an individual afflicted with a mood disorder as defined herein, in the process of identifying and characterizing the relevant gene. For example, the inventors are able to make use of the previously identified apparent association between trinucleotide repeat expansions (TRE) within the human genome and the phenomenon of anticipation in mood disorders (Lindblad et al. (1995), Neurobiology of Disease 2. pp 55-62 and O'Donovan et al. (1995), Nature Genetics 1Q pp 380-381) to screen for TRE's in the selected YAC clones in order to identify candidate genes in the region of interest on human chromosome18. A variety of other known procedures can also be applied to the said YAC clones to identify the candidate gene as discussed below.

Accordingly, in a first aspect the present invention comprises the use of an 8.9 cM region of human chromosome 18q disposed between polymorphic markers D18S68 and D18S979 or a fragment thereof for identifying at least one human gene, including mutated and polymorphic variants thereof, which is associated with mood disorders or related disorders as defined above. As will be described below, the present inventors have identified this candidate region of chromosome 18q for such a gene, by analysis of co-segregation of bipolar disease in family MAD31 with 12 STR polymorphic markers previously located between D18S51 and D18S61 and subsequent LaD score analysis. Particular YACs covering the candidate region which may be used in accordance with the present invention are 961.h-9, 942-c.3, 766-f-12, 731-c- 7, 907.e.1, 752-g-8 and 717-d-3, preferred ones being 961h-9, 766.f.12 and 907-e.1 since these have the minimum tiling path across the candidate region suitable YAC clones for use are those having an artificial chromosome spanning the refined candidate region between D18S68 and D18S979.

There are a number of methods which can be applied to the candidate regions of chromosome 18q as defined above, whether or not present in a YAC, to identify a candidate gene or genes associated with mood disorders or related disorders. For example, as aforesaid, there is an apparent association between the extent of trinucleotide repeat expansions (TRE) in the human genome and the presence of mood disorders.

Accordingly, in a third aspect the present invention comprises a method of identifying at least one human gene, including mutated and polymorphic variants thereof, which is associated with a mood disorder or related disorder as defined herein which comprises detecting nucleotide triplet repeats in the region of human chromosome 18q disposed between polymorphic markers D18S68 and D18S979.

An alternative method of identifying said gene or genes comprises fragmenting a YAC clone comprising a portion of human chromosome 18q disposed between polymorphic markers D18S60 and D18S61, for example one or more of the seven aforementioned YAC clones, and detecting any nucleotide triplet repeats in said fragments, in particular repeats of CAG or CTG. Nucleic acid probes comprising at least 5 and preferably at least 10 CTG and/or CAG triplet repeats are a suitable means of detection when appropriately labelled. Trinucleotide repeats may also be determined using the known RED (repeat expansion detection) system (Shalling et al. (1993), Nature Genetics˜pp 135-139).

In a fourth embodiment the invention comprises a method of identifying at least one gene, including mutated and polymorphic variants thereof, which is associated with a mood disorder or related disorder and which is present in a YAC clone spanning the region of human chromosome 18q between polymorphic markers D18S60 and D18S61, the method comprising the step of detecting the expression product of a gene incorporating nucleotide triplet repeats by use of an antibody capable of recognizing a protein with anamino acid sequence comprising a string of at least 8, but preferably at least 12, continuous glutamine residues. Such a method may be implemented by sub-cloning YAC DNA, for example from the seven aforementioned YAC clones, into a human DNA expression library. A preferred means of detecting the relevant expression product is by use of a monoclonal antibody, in particular mAB 1 C2, the preparation and properties of which are described in International Patent.

Application Publication No WO 97/17445.

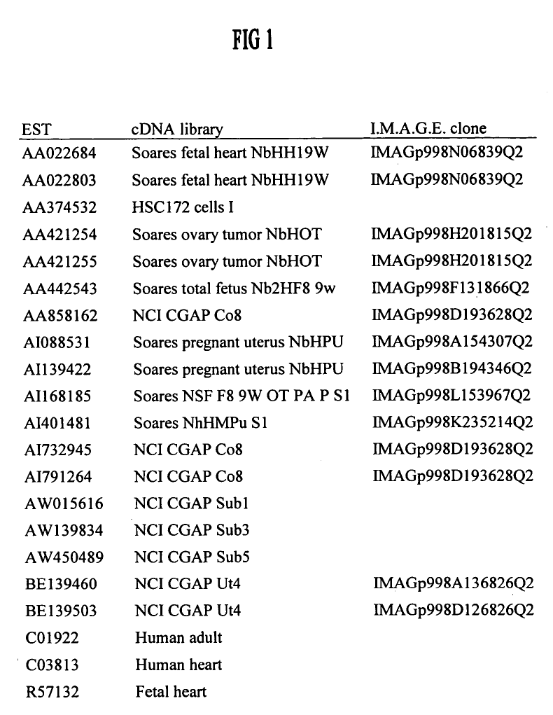

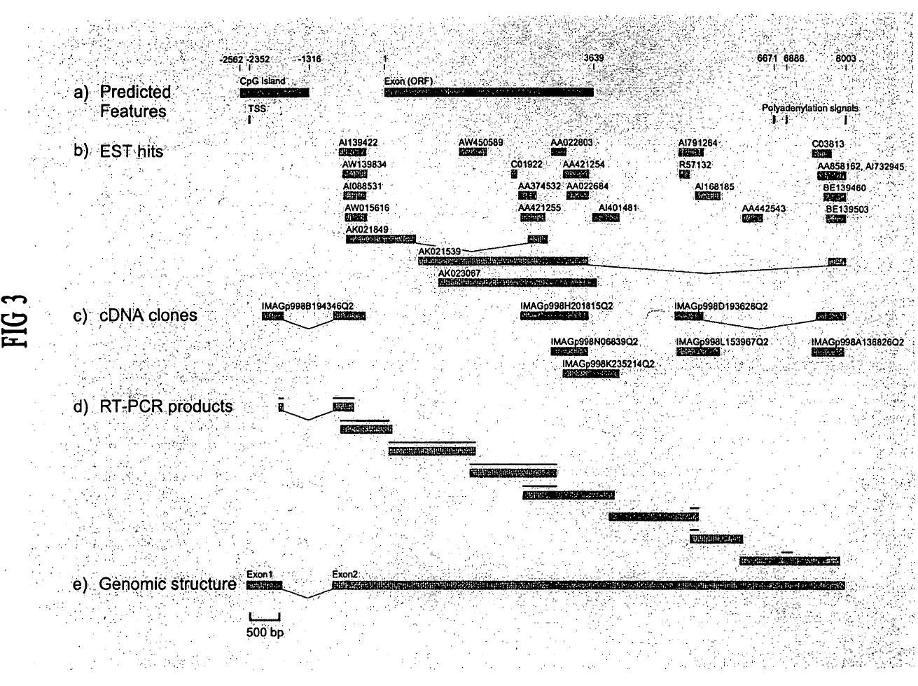

Further embodiments of the present invention relate to methods of identifying the relevant gene orgenes which involve the sub-cloning of YAC DNA as defined above into vectors such as BAC (bacterial artificial chromosome) or PAC (P1 or phage artificial chromosome) or cosmid vectors such as exon-trap cosmid vectors. The starting point for such methods is the construction of a contig map of the region of human chromosome 18q between polymorphic markers D18S60 and D18S61. To this end the present inventors have sequenced the end regions of the fragment of human DNA in each of the seven aforementioned YAC clones and these sequences are disclosed herein. Following sub-cloning of YAC DNA into other vectors as described above, probes comprising these end sequences or portions thereof, in particular those sequences shown in FIGS. 1 to 11 herein, together with any known sequenced tagged site (STS) in this region, as described in the YAC clone contig shown herein, as can be used to detect overlaps between said sub-clones and a contig map can be constructed. Also the known sequences in the current YAC contig can be used for the generation of contig map sub-clones.

One route by which a gene or genes which is associated with a mood disorder or associated disorder can be identified is by use of the known technique of exon trapping. This is an artificial RNA splicing assay, most often making use in current protocols of a specialized exon-trap cosmid vector. The vector contains an artificial mini-gene consisting of a segment of the SV40 genome containing an origin of replication and a powerful promoter sequence, two splicing-competentexons separated by an intron which contains a multiple cloning site and an SV40 polyadenylation site.

The YAC DNA is sub-cloned in the exon-trap vector and the recombinant DNA is transfected into a strain of mammalian cells. Transcription from the SV40 promoter results in an RNA transcript which normally splices to include the two exons of the minigene. If the cloned DNA itself contains a functional exon, it can be spliced to the exons present in the vector's minigene. Using reverse transcriptase a cDNA copy can be made and using specific PCR primers, splicing events involving exons of the insert DNA can be identified. Such a procedure can identify coding regions in the YAC DNA which can be compared to the equivalent regions of DNA from a person afflicted with a mood disorder or related disorder to identify the relevant gene.

Accordingly, in a fifth aspect the invention comprises a method of identifying at least one human gene, including mutated variants and polyrnorphisms thereof, which is associated with a mood disorder or related disorder which comprises the steps of:
(1) transfecting mammalian cells with exon trap cosmid vectors prepared and mapped as described above;
(2) culturing said mammalian cells in an appropriate medium;
(3) isolating RNA transcripts expressed from the SV40 promoter;
(4) preparing cDNA from said RNA transcripts;
(5) identifying splicing events involving exons of the DNA sub-cloned into said exon trap cosmid vectors to elucidate positions of coding regions in said sub-cloned DNA;
(6) detecting differences between said coding regions and equivalent regions in the DNA of an individual afflicted with said mood disorder or related disorder; and
(7) identifying said gene or mutated orpolymorphic variant thereof which is associated with said mood disorder or related disorders.

As an alternative to exon trapping the YAC DNA may be sub-cloned into BAC, PAC, cosmid or other vectors and a contig map constructed as described above. There are a variety of known methods available by which the position of relevant genes on the sub-cloned DNA can be established as follows:
(a) cDNA selection or capture (also called direct selection and cDNA selection): this method involves the forming of genomic DNA/cDNA heteroduplexes by hybridizing a cloned DNA (e.g. an insert of a YAC DNA), to a complex mixture of cDNAs, such as the inserts of all cDNA clones from a specific (e.g. brain) cDNA library. Related sequences will hybridize and can be enriched in subsequent steps using biotin-streptavidine capturing and PCR (or related techniques);
(b) hybridization to mRNA/cDNA: a genomic clone (e.g. the insert of a specific cosmid) can be hybridized to a Northern blot of mRNA from a panel of culture cell lines or against appropriate (e.g. brain) cDNA libraries. A positive signal can indicate the presence of a gene within the cloned fragment;
(c) CpG island identification: CpG or HTF islands are short (about 1 kb) hypomethylated GC-rich (>60%) sequences which are often found at the 5′ ends of genes. CpG islands often have restriction sites for several rare-cutter restriction enzymes. Clustering of rare-cutter restriction sites is indicative of a CpG island and therefore of a possible gene. CpG islands can be detected by hybridization of a DNA clone to Southern blots of genomic DNA digested with rare-cutting enzymes, or by island-rescue PCR (isolation of CpGislands from YACs by amplifying sequences between islands and neighbouring Alu-repeats);
(d) zoo-blotting: hybridizing a DNA clone (e.g. the insert of a specific cosmid) at reduced stringency against a Southern blot of genomic DNA samples from a variety of animal species. Detection of hybridization signals can suggest conserved sequences, indicating a possible gene. Accordingly, in a sixth aspect the invention comprises a method of identifying at least one human gene including mutated and polymorphic variants thereof which is associated with a mood disorder or related disorder which comprises the steps of:
(1) sub-cloning the YAC DNA as described above into a cosmid, BAC, PAC or other vector;
(2) using the nucleotide sequences shown in any one of FIGS. 1 to 11 or any other sequenced tagged site (STS) in this region as in the YAC clone contig described herein, or part thereof consisting of not less than 14 contiguous bases or the complement thereof, to detect overlaps amongst the sub-clones and construct a map thereof;
(3) identifying the position of genes within the sub-cloned DNA by one or more of CpG island identification, zoo-blotting, hybridization of the sub-cloned DNA to a cDNA library or a Northern blot of mRNA from a panel of culture cell lines;
(4) detecting differences between said genes and equivalent region of the DNA of an individual afflicted with a mood disorder or related disorder; and
(5) identifying said gene which is associated with said mood disorders or related disorders.

If the cloned YAC DNA is sequenced, computer analysis can be used to establish the presence of relevant genes. Techniques such as homology searching and exon prediction may be applied.

Once a candidate gene has been isolated in accordance with the methods of the invention more detailed comparisons may be made between the gene from a normal individual and one afflicted with a mood disorder such as a bipolar spectrum disorder. For example, there are two methods, described as “mutation testing”, by which a mutation or polymorphism in a DNA sequence can be identified. In the first the DNA sample may be tested for the presence or absence of one specific mutation but this requires knowledge of what the mutation might be. In the second a sample of DNA is screened for any deviation from a standard (normal) DNA. This latter method is more useful for identifying candidate genes where a mutation is not identified in advance. In addition the following techniques may be further applied to a gene identified by the above-described methods to identify differences between genes from normal or healthy individuals and those afflicted with a mood disorder or related disorder:
(a) Southern blotting techniques: a clone is hybridized to nylon membranes containing genomic DNA digested with different restriction enzymes of patients and healthy individuals. Large differences between patients and healthy individuals can be visualized using a radioactive labelling protocol;
(b) heteroduplex mobility in polyacrylamide gels: this technique is based on the fact that the mobility of heteroduplexes in non-denaturing polyacrylamide gels is less than the mobility of homoduplexes. It is most effective for fragments under 200 bp;
(c) single-strand conformational polymorphism analysis (SSCP or SSCA): single stranded DNA folds up to form complex structures that are stabilized by weak intramolecular bonds.

The electrophoretic mobilities of these structures on non-denaturing polyacrylamide gels depends on their chain lengths and on their conformation;
(d) chemical cleavage of mismatches (CCM) : a radiolabelled probe is hybridized to the test DNA, and mismatches detected by a series of chemical reactions that cleave one strand of the DNA at the site of the mismatch. This is a very sensitive method and can be applied to kilobase-length samples;
(e) enzymatic cleavage of mismatches: the assay is similar to CCM, but the cleavage is performed by certain bacteriophage enzymes.
(f) denaturing gradient gel electrophoresis: in this technique, DNA duplexes are forced to migrate through an electrophoretic gel in which there is a gradient of increasing amounts of a denaturant (chemical or temperature). Migration continues until the DNA duplexes reach a position on the gel wherein the strands melt and separate, after which the denatured DNA does not migrate much further. A single base pair difference between a normal and a mutant DNA duplex is sufficient to cause them to migrate to different positions in the gel;
(g) direct DNA sequencing.

It will be appreciated that with respect to the methods described herein, in the step of detecting differences between coding regions from the YAC and the DNA of an individual afflicted with a mood disorder or related disorder, the said individual may be anybody with the disorder and not necessary a member of family MAD31.

In accordance with further aspects the present invention provides an isolated human gene and variants thereof associated with a mood disorder or related disorder and which is obtainable by any of the above described methods, an isolated human protein encoded by said gene and a cDNA encoding said protein.

Once a gene has been identified a number of methods are available to determine the function of the encoded protein. These methods are described by Eisenberg et al (Nature vol. 15, June 2000) and is herein incorporated by reference. One method involves a computational method that reveals functional linkages from genome sequences and is called the gene neighbor metho. If in several genomes the genes that encode two proteins are neighbors on the chromosome, the proteins tend to be functionally linked. This method can be powerful in uncovering functional linkages in prokaryotes, where operons are common, but also shows promise for analysing interacting proteins in eukaryotes.

### EXAMPLE

### Example 1

A: Triplet Repeat Isolation

CCG/CGG YAC fragmentation vectors were constructed by cloning blunted (CCG)₁₀/(CGG)₁₀ adapters into the blunted SphI site of the previously described pDV1 basic vector(Del-Favero et al 1999). Sequencing determined that fragmentation vectors pDVCCG and pDVCGG have the adapter sequence in a 5′-(CCG)₁₀-3′ and a 5′-(CGG)₁₀-3′ orientation respectively.

Using these vectors, CCG/CGG repeats and flanking sequences were isolated by YAC fragmentation as described(Del-Favero et al 1999).

B: Characterisation of Structure of the NCAG1 Gene.

I.M.A.G.E. Consortium [LLNL] cDNA Clones(Lennon et al 1996) IMAGp998A 136826Q2, IMAGp998A 154307Q2, IMAGp998B194346Q2, IMAGp998D126826Q2, IMAGp998DI93628Q2, IMAGp998F131866Q2, IMAGp998H201815Q2, IMAGp998K235214Q2, IMAGp998L153967Q2 and IMAGp998N06839Q2 were ordered at RZPD Deutsches Ressourcenzentrum fur Genomforschung GmbH (Heubnerweg 6, 14059 Berlin-Charlottenburg, Germany). Cultures starting from single colonies were grown and plasmids were prepared by the Wizard Plus SV Minipreps DNA Purification System (Promega, Madison, Wis.). DNA sequencing was performed with the dideoxynucleotide sequencing method using a DNA sequencing kit (Perkin-Elmer, Foster, Calif.) and analysed by an ABI PRISM 377 DNA Sequencer (Perkin-Elmer, Foster, Calif.) or an ABI PRISM 3700 DNA Analyser (Perkin-Elmer, Foster, Calif.).

For the RT-PCR reactions, mRNA from SHSY-5Y cells was prepared using the μMACS mRNA Isolation Kit (Miltenyi Biotec, Bergisch Gladbach, Germany). After DNAseI treatment (Promega, Madison, Wis.), the RT reaction was primed with oligo(dT) primers and performed with Superscript Preamplification System for First Strand cDNA synthesis (GibcoBRL, N.V. Life Technologies, Merelbeke, Belgium). Fs-cDNA was used in long-range PCR reactions with TaKaRa LA Taq (Takara Shuzo Co., Otsu, Shiga, Japan). PCR products were reamplified with nested primers and sequenced as described above.

C: Characterisation of the Expression Pattern of the NCAG1 Gene.

Genepool cDNA (Invitrogen, Carlsbad, Calif.) from brain, fetal brain, placenta, liver, testis and lung was used as a cDNA mapping panel. The Human Brain Multiple Tissue Northern (MTN) Blot IV (Clontech, Palo Alto, Calif.) was used for radioactive hybridisation in accompanying ExpressHyb solution according to the instructions of the manufacturer. A zooblot was prepared by digesting 10 μg genomic DNA to completion with HindIII, running it on a TAE 1% agarose gel and performing a Southern blot. A PCR product containing the ORF of the NCAG1 gene was radioactively labelled and hybridised at 65° C.

D: Mutation Analysis of the NCAG1 Gene.

Overlapping PCR products of approximately 600 bp were generated and sequenced as described above. Both identified polymorphisms were detected by digesting the PCR product with Hinfl and electrophoresing the fragments on precast ExcelGel gels on a Multiphor II electrophoresis system (Amersham Pharmacia Biotech AB, Uppsala, Sweden)

E: CCG/CGG YAC Fragmentation

CCG/CGG YAC fragmentation was applied to YACs 961h9, 766fl2 and 907el(Goossens et al 2000). Size determination by Pulsed Field Gel Electrophoresis (PFGE) and Southern blot hybridisation resulted in 33 sets of equally sized fragmented YAC clones. Sequencing of 112 fragmented YAC ends identified seven (out of 33) sets of fragmented. YACs with identical end sequences resulting from a specific homologous recombination. One set (CCG7) was the result of fragmentation in the (CGG)₆ repeat in the 5′ UTR of the CAP2 gene (GenBank acc. No L40377). A second set (CCG6) contained a (CCG)₂ repeat and a third (CCG4) an imperfect CCCCG repeat. The triplet repeat in the 5′ UTR of the CAP2 gene was already shown not to be associated with BP disorder(Goossens et al 2000). The size of CCG4 was analyzed in 12 BP and 12 UP patients, but only one allele was detected. The size of CCG6 was not analyzed since it was to small to be polymorphic.

In depth analysis showed that three (CCG3, GenBank acc No . . . ; CCG4, GenBank acc No . . . and CCG6, GenBank acc No . . . ) of the seven sequences had high CG content (70-80%) and high CpG content (15-20 CpGs in 200 bp) but no additional CCG/CGG repeats were found. Primer pairs for these potential CpG islands were used to determine their position on the YAC contig (FIG. 1). BLASTN analysis(Altschul et al 1990) resulted for both CCG4 and CCG6 in hits with sequences of RPCI-11 BACs. CCG4 gave a hit in a contig of 27150 bp of the working draft sequence of RPCI-11 BAC 29013 (GenBank acc No AC022662, GI: 7249117). CCG6 was part of the complete sequence of RPCI-11 BAC 793J2 (GenBank acc No AC009802).

F: Identification and in Silico Characterisation of NCAG1 Gene.

To find genes possibly associated with the potential CpG islands CCG4 and CCG6, their surrounding BAC sequences were analysed using bioinformatic tools. Hence the 27150 bp contig of BAC 29013 and the complete sequence of BAC 793J2 were sent for analysis to the Rummage High-Throughput Sequence Annotation Server (http://gen100.imb-jena.de/rummage/index.html).

First, LCP(Huang 1994) and CPG(Larsen et al 1992) recognized CpG islands containing CCG4 and CCG6 of 1.2 kb and 0.4 kb respectively, confirming their potential role as CpG islands.

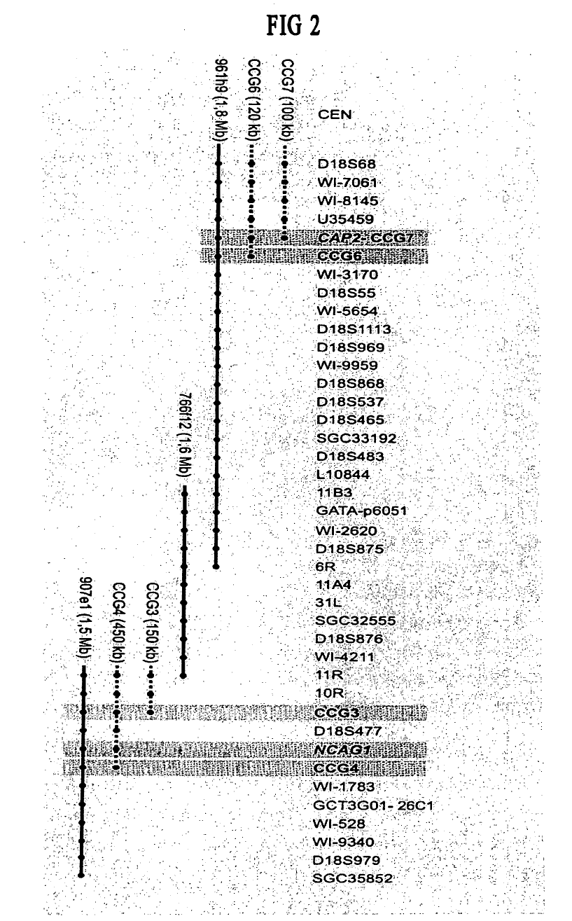

In a next step, exon prediction programs Grail(Uberbacher & Mural 1991) and Genscan(Burge & Karlin 1997) both predicted the presence of a 3639 bp exon, 1.5 kb downstream of the 1.2 kb large CpG island containing CCG4. This predicted exon contains an open reading frame (ORF) which starts at an ATG start codon with an almost perfect Kozak sequence and ends with a TAA stop codon. Other predicted features are a transcription start site (TSS) at 2352 bp upstream of the ORF (score 76.6 by Proscan(Prestridge 1995)) and polyadenylation signals at 3032, 3247, 4364, 5338 and 8266 downstream of the ORF (respective scores of 4.79, 3.83, 4.94, 4.93 and 6.27 by PolyAH(Salamov & Solovyev 1997)) (FIG. 2a).

BLASTN(Altschul et al 1990) alignment searches to sequences of dbEST revealed significant homology (>97%) to 21 human ESTs (Table 1, FIG. 2b). TBLASTX(Altschul et al 1997) searches of the Genbank non-redundant database (nr) with the ORF showed extensive homology on protein level with SART-2 (Genbank Acc No NP_{—}037484), a squamous cell carcinoma antigen recognized by T-cells(Nakao et al 2000). Weaker homology was found with a series of sulfotransferases. Analysis of the 1212 long aminoacid sequence of the translated ORF by SMART (Simple Modular Architecture Research Tool, V3.1)(Schultz et al 2000) did not result in any known domains apart from a cleavable signal peptide at position 1-20 and two transmembrane segments at positions 771-791 and 800-820. Interpro reporterd no significant hits, although BLASTP(Altschul et al 1997) of the Prodom database showed homology between the NCAGI gene and the chondroitin-6-sulfotransferase domain (Prodom Acc No PD042460)

G: Characterisation of the Structural Organisation of the NCAG1 Gene.

Based on the BLASTN EST hits I.M.A.G.E. Consortium [LLNL] cDNA Clones(Lennon et al 1996) were ordered and sequenced. The sequences alligned with the genomic sequence in the presumed 5′ UTR (untranslated region), the ORF and the presumed 3′ UTR, indicating that these sequences are indeed transcribed (FIG. 2c). Alignment of the sequence of IMAGp998B194346Q2 with the genomic sequence showed that a 865 bp fragment was missing in the cDNA. A detailed analysis of the flanking sequences revealed the presence of consensus acceptor and donor splice sites, confirming that this fragment is probably an intron. Also clone IMAGp998D193628Q2 missed a fragment of 1.9 kb when compared to the genomic sequence, but consensus splice sites were absent. Two clones, IMAGp998D193628Q2 and IMAGp998A136826Q2, terminated exactly at the predicted polyadenylation signal, 4.4 kb downstream of the ORF. Sequences of clones IMAGp998A154307Q2, IMAGp998D126826Q2 and IMAGp998F131866Q2 did not align with the genomic sequence and were not analysed further.

Since cDNA clone sequencing did not result in a continuous sequence of the transcript, primers were designed and used for RT-PCR experiments. Sequencing of different overlapping RT-PCR products confirmed the presence of a transcript of at least 9 kb, containing the ORF of the predicted exon, linked to the presumed 5′ and 3′ sequences (FIG. 2d). The 5 prime intron of 865 bp was confirmed and the 3′ UTR was extended till the predicted polyadenylation signal, 4.4 kb downstream of the ORF.

H: Characterisation of the Expression Pattern of the NCAG1 Gene.

To investigate the expression profile of the NCAG1 gene, a long-range PCR spanning the ORF was optimised on genomic DNA and applied on a cDNA mapping panel. This showed that the fragment was present in cDNA from brain, fetal brain, placenta and liver but could not be detected in cDNA from testis and lung. More detailed information on the expression in the brain was obtained by Northern blot hybridisation showing expression of a >9.5 kb transcript in all investigated tissues (lung, placenta, small intestine, liver, kidney, skeletal muscle, heart, brain, uterus, trachea, thyroid, stomach, spinal cord, prostate, mammary gland, lymph node, brain (whole), bladder, adrenal gland, amygdala, caudate nucleus, corpus callosum, hippocampus, substantia nigra, thalamus and total brain).

Stringent Zooblot hybridisation experiments showed the presence of homologous sequences in the genomic DNA of other mammals like dog, pig, mouse, donkey, horse and sheep.

I: Mutation Analysis of the NCAG1 Gene.

Since this novel CpG-associated gene is brain-expressed and located in the chromosome 18q21.3-q23 BP candidate region, a mutation analysis of the ORF was performed on 3 patients and 1 escapee of the chromosome 18 linked family MAD31. In this way two single nucleotide polymorphisms were identified. The first is a C to T transition on position 2017 of the ORF, changing aminoacid (AA) 673 from proline to serine. This polymorphism was only found in the healthy control. The second polymorphism was found in all three patients. It was also a C to T transition, located at position 2824 and changing the 942 AA from proline to serine. Analysis of this polymorphism in family MAD31 showed that the T-allele was present on the disease haplotype.

Both polymorphisms were analysed in an association study on 92 BP patients and 92 age, sex and ethnicity matched controls by PCR-RFLP analysis. The P673S polymorphism turned out to be a frequent polymorphism with both alleles roughly equally present. The P942S polymorphism however was found to be a rare polymorphism, with the T allele only present in 3 BP patients and in 2 controls. Statistical analysis showed the control population was in Hardy-Weinberg equilibrium for both polymorphisms. No alleles, genotypes or haplotypes were found to be associated to BP disorder.

Since triplet repeat fragmentation was proven to be a valid method for the region specific isolation of triplet repeats(Goossens et al 2000), we applied it to the chromosome 18q21.33-q23 BP candidate region for the isolation of CCG/CGG repeats. Therefore, we first had to construct a new set of fragmentation vectors, pDVCCG and pDVCGG. Fragmentation experiments with these vectors resulted in transformation and fragmentation efficiencies in the same range as obtained with the CAG/CTG fragmentation vectors pDVCAG and pDVCTG (data not shown). Application of CCG/CGG fragmentation to YAC 961h9 resulted in the isolation of the (CGG)₆ repeat in the 5′ UTR of CAP2. This repeat is adjacent to the (CAG)₆ repeat previously reported(Goossens et al 2000). There, it was shown that this (CGG)₆(CAG)₆ repeat is polymorphic but not expanded in BP cases nor associated with BP disorder. Taken together, the CCG/CGG YAC fragmentation data does not support CCG/CGG repeats as disease causing agents in chromosome 18q21.33-q23 linked BP disorder. On the other hand, fragmentation experiments resulted in three sequences (CCG3, CCG4 and CCG6) with high CG (70-80%) and CpG content but containing no CCG/CGG repeat. CpG islands are usually defined as regions of DNA of more than 200 bases that have a CG content above 50% and a ratio of observed versus expected CpGs close to that statistically expected. Therefore, CCG3, CCG4 and CCG6 can be considered as potential CpG islands. Analysis of surrounding sequences of CCG4 and CCG6 with LCP(Huang 1994) and CPG(Larsen et al 1992) confirmed that the fragmentation occurred in both cases indeed in a CpG island. Since CpG islands are strongly associated with genes, more specifically housekeeping and widely expressed genes, these three sequences are likely to be located near this class of genes.

In the search for genes possibly associated with the isolated CpG islands, exon prediction programs Grail(Uberbacher & Mural 1991) and Genscan(Burge & Karlin 1997) both predicted the presence of a 3.6 kb exon downstream of the largest CpG island isolated. Two facts argued strongly against a false positive prediction. The first was that this two programs, based on different models, predicted exactly the same exon. The second was the mere presence in genomic DNA of this ORF continuing for 3.6 kb and starting with a Kozak consensus ATG. Additional evidence that this exon was indeed transcribed was found in the fact that a series of ESTs had very high homologies (97-100%) with sequences in and surrounding the ORF. In a next step, this evidence was extended by sequencing of the cDNA clones from which the ESTs originated. The EST sequences were prolonged and corrected and the homologies increased to 99-100%. The fact that the cDNA clones originated from different cDNA libraries (Table 1) indicated that the gene was expressed in different tissues. RT-PCR and northern blot experiments resulted in the final confirmation that this ORF was widely expressed, a usual characteristic of a CpG-associated gene.

cDNA clone sequencing resulted in complete sequence of seven human cDNA clones aligning with NCAG1. In two cases a piece of genomic DNA was missing in the cDNA sequence. Clone IMAGp998B194346Q2 lacked a 865 bp fragment (FIG. 2c). Since this fragment was flanked by splice donor and acceptor consensus sequences, and since the fragment was also missing in the RT-PCR products, enough evidence was gathered to call it an intron. Clone IMAGp998D193628Q2 also missed a 1.4 kb fragment compared to the genomic sequence. In this case no consensus splice sites were present. Moreover cDNA clones IMAGp998L153967Q2 and IMAGp998A136826Q2 contain sequences that are located in the missing fragment of IMAGp998D193628Q2 (FIG. 2c). This data together with the fact that EST AA442543 is located entirely in the missing fragment (FIG. 2b) and the presence of this fragment in the RT-PCR products (FIG. 2d) indicate that this fragment might rather be an artifact than an intron.

EST-homologies and cDNA clone sequencing proved that a series of cDNA clones terminated at a predicted polyadenylation signal, 4.3 kb downstream of the ORF or 10.3 kb downstream of the predicted TSS. If the 5 prime intron of 865 bp is taken into account, the size of transcript will be 9.5 kb, which is the size of the transcript recognized in the Northern blot experiment.

On protein level, a cleavable signal peptide and two transmembrane domains are predicted. If this is correct, both N-terminal and C-terminal sides will be at the same side of the membrane in which it is embedded. The strong homology with the SART-2 protein is significant, but it does not add more clues as to potential functions of the novel protein.

The 2824T allele, present on the disease haplotype in the chromosome 18 linked family MAD31, is a very rare allele with a frequency of 0.03. Therefore statistical analysis in an association sample loses a lot of its strength, leaving the possibility that this allele confers an increased risk for BP disorder.

References

The following references are herein expressly incorporated by reference:
1. Altschul S F, Gish W, Miller W, Myers E W, Lipman D J. 1990. Basic local alignment search tool. J. Mol. Biol. 215:403-10
2. Altschul S F, Madden T L, Schaffer A A, Zhang J, Zhang Z, Miller W, Lipman D J. 1997. Gapped BLAST and PSI-BLAST: a new generation of protein database search programs. Nucleic Acids Res. 25(17):3389-402
3. Burge C, Karlin S. 1997. Prediction of complete gene structures in human genomic DNA. J. Mol. Biol. 268(1):78-94
4. Del-Favero J, Goossens D, Van den Bossche D, Van Broeckhoven C. 1999. YAC fragmentation with repetitive and single-copy sequences: detailed physical mapping of the presenilin 1 gene on chromosome 14. Gene 229:193-201
5. Del Favero J, Goossens D, De Jonghe P, Benson K, Michalik A, Van den B D, Horwitz M, Van Broeckhoven C. 1999. Isolation of CAG/CTG repeats from within the chromosome 2p21-p24 locus for autosomal dominant spastic paraplegia (SPG4) by YAC fragmentation. Hum. Genet. 105(3):217-25
6. Eichhammer P, Walz A, Mengling T, Scholer A, Putzhammer A, Rohrmeier T, Aigner J M, Klein H E, Schlegel J. 1998. Detection of polymorphic triplet repeats in the genomes of patients suffering from bipolar affective disorder. Int. J. Mol. Med. 1(6):989-93
7. Goossens D, Villafuerte S, Tissir F, Van Gestel S, Claes S, Souery D, Massat I, Van den Bossche D, Van Zand K, Mendlewicz J, Van Broeckhoven C, Del-Favero J. 2000. No evidence for the involvement of CAG/CTG repeats from within 18q21.33-q23 in bipolar disorder. Eur. J. Hum. Genet. 8(5):385-8
8. Huang X. 1994. An algorithm for identifying regions of a DNA sequence that satisfy a content requirement. Comput. Appl. Biosci. 10(3):219-25
9. Kaushik N, Malaspina A, de Belleroche J. 2000. Characterization of trinucleotide- and tandem repeat-containing transcripts obtained from human spinal cord cDNA library by high-density filter hybridization. DNA Cell Biol. 19(5):265-73
10. Kleiderlein J J, Nisson P E, Jessee J, Li W B, Becker K G, Derby M L, Ross C A, Margolis R L. 1998. CCG repeats in cDNAs from human brain. Hum. Genet. 103(6):666-73
11. Larsen F, Gundersen G, Lopez R, Prydz H. 1992. CpG islands as gene markers in the human genome. Genomics 13(4):1095-107
12. Lennon G, Auffray C, Polymeropoulos M, Soares M B. 1996. The I.M.A.G.E. Consortium: an integrated molecular analysis of genomes and their expression. Genomics 33(1):151-2
13. Mangel L, Ternes T, Schmitz B, Doerfler W. 1998. New 5′-(CGG)n-3′ repeats in the human genome. J. Biol. Chem. 273(46):30466-71
14. Margolis R L, McInnis M G, Rosenblatt A, Ross C A. 1999. Trinucleotide repeat expansion and neuropsychiatric disease. Arch. Gen. Psychiatry 56(11):1019-31
15. McInnis M G, McMahon F J, Chase G A, Simpson S G, Ross C A, DePaulo J R J. 1993. Anticipation in bipolar affective disorder. Am. J. Hum. Genet. 53:385-90
16. Nakao M, Shichijo S, Irnaizumi T, Inoue Y, Matsunaga K, Yamada A, Kikuchi M, Tsuda N, Ohta K, Takamori S, Yamana H, Fujita H, Itoh K. 2000. Identification of a gene coding for a new squamous cell carcinoma antigen recognized by the CTL. J. Immunol. 164(5):2565-74
17. Nylander P O, Engstrom C, Chotai J, Wahistrom J, Adolfsson R. 1994. Anticipation in Swedish families with bipolar affective disorder. J. Med. Genet. 31:686-9
18. Prestridge D S. 1995. Predicting Pol II promoter sequences using transcription factor binding sites. J. Mol. Biol. 249(5):923-32
19. Salamov A A, Solovyev V V. 1997. Recognition of 3′-processing sites of human mRNA precursors. Comput. Appl. Biosci. 13(1):23-8
20. Schultz J, Copley R R, Doerks T, Ponting C P, Bork P. 2000. SMART: a web-based tool for the study of genetically mobile domains. Nucleic Acids Res. 28(1):231-4
21. Uberbacher E C, Mural R J. 1991. Locating protein-coding regions in human DNA sequences by a multiple sensor-neural network approach. Proc. Natl. Acad Sci. U.S.A 88(24):11261-5
22. Van Broeckhoven C, Verheyen G. 1999. Report of the chromosome 18 workshop. Am. J. Med. Genet. 88(3):263-70
23. Verheyen G R, Villafuerte S M, Del-Favero J, Souery D, Mendlewicz J, Van Broeckloven C, Raeymaekers P. 1999. Genetic refinement and physical mapping of a chromosome 18q candidate region for bipolar disorder. Eur. J. Hum. Genet. 7(4):427-34

1. An isolated nucleic acid comprising the nucleotide sequence of SEQ ID N0: 1.

2. An isolated nucleic acid consisting essentially of the nucleotide sequence of SEQ ID N0: 1.

3. An isolated nucleic acid for comprising a nucleotide sequence that encodes the amino acid sequence of SEQ ID N0: 2.

4. An isolated nucleic acid comprising the nucleotide sequence of SEQ ID N0: 3.

5. An isolated nucleic acid consisting essentially of the nucleotide sequence of SEQ ID N0: 3.

6. An isolated nucleic acid consisting of the nucleotide sequence of SEQ ID N0: 1 or a contiguous fragment thereof wherein said isolated nucleic acid encodes a polypeptide having biological activity of bipolar disorder protein.

7. An isolated nucleic acid that hybridizes under high stringency conditions to a nucleic acid having a sequence complementary to the nucleotide sequence of SEQ ID N0: 1, wherein said isolated nucleic acid encodes a polypeptide having biological activity.

8. An isolated nucleic acid that encodes a polypeptide having the biological activity, said isolated nucleic acid consisting of a nucleotide sequence that is at least 90% identical to the nucleotide sequence of SEQ ID N0: 1.

9. An isolated nucleic acid consisting of the nucleotide sequence of SEQ ID N0: 3 or a contiguous fragment thereof wherein said isolated nucleic acid encodes a polypeptide having biological activity.

10. An isolated nucleic acid that hybridizes under high stringency conditions to a nucleic acid having a sequence complementary to the nucleotide sequence of SEQ ID N0: 3, wherein said isolated nucleic acid encodes a polypeptide having the biological activity.

11. An isolated nucleic acid that encodes a polypeptide having the biological activity;, said isolated nucleic acid consisting of a nucleotide sequence that is at least 90% identical to the nucleotide sequence of SEQ ID N0: 3.

12. Isolated and substantially purified protein encoded by the nucleic acid of claim 6.

13. Isolated and substantially purified viral inhibitory protein 1 and 2 encoded by the nucleic acid of claim 9.

14. Isolated and substantially purified viral inhibitory protein having the amino acid sequence of SEQ ID N0: 2.

15. Isolated and substantially purified protein having an amino acid sequence that is at least 90% identical to the sequence of SEQ ID N0:2.

16. Isolated and substantially purified protein having an amino acid sequence that is at least 90% identical to the sequence of SEQ ID N0:4.

17. Isolated and substantially purified protein having an amino acid sequence that is at least 90% identical to the sequence of SEQ ID N0: 4.

18. A vector comprising the nucleic acid of claim 1.

19. A vector comprising the nucleic acid of claim 4.

20. A vector comprising the nucleic acid of claim 6 operable linked to an expression control sequence.

21. A host cell comprising the nucleic acid of claim 6.

22. A host cell comprising the vector of claim 20.

23. A method of making protein 1 and 2 comprising:
a) introducing the nucleic acid of claim 6 into a host cell;
b) maintaining said host cell under conditions whereby said nucleic acid is expressed to protein;
c) recovering said protein.

24. A method of making protein comprising:
a) introducing the nucleic acid of claim 9 into a host cell;
b) maintaining said host cell under conditions whereby said nucleic acid is expressed to produce protein;
c) recovering said protein.

25. A method of making protein comprising:
a) introducing the nucleic acid of claim 16 into a host cell;
b) maintaining said host cell under conditions whereby said nucleic acid is expressed to produce viral inhibitory protein;
c) recovering said protein.

26. A composition comprising purified protein and a carrier.

27. The composition according to claim 26 which further comprises viral inhibitory protein 2.
